(19) United States
(12) Patent Application Publication
(10) Pub. No.: US 2014/0051894 A1
(43) Pub. Date: Feb. 20, 2014

(54) Silica Enhanced Support for Hydrogenation Catalysts and Processes for Producing Ethanol

(71) Applicants: Heiko Weiner, Pasadena, TX (US); Radmila Wollrab, Pasadena, TX (US); Zhenhua Zhou, Houston, TX (US)

(72) Inventors: Heiko Weiner, Pasadena, TX (US); Radmila Wollrab, Pasadena, TX (US); Zhenhua Zhou, Houston, TX (US)

(73) Assignee: CELANESE INTERNATIONAL CORPORATION, Irving, TX (US)

(21) Appl. No.: 13/586,311

(22) Filed: Aug. 15, 2012

(51) Int. Cl. B01J 23/02; B01J 23/04; B01J 23/78; B01J 23/58; C07C 29/149; B01J 23/14

(52) U.S. Cl. 568/885; 502/242; 502/243; 502/250

(57) ABSTRACT

The present invention relates to a catalyst. The catalyst is used for converting acetic acid to ethanol. The catalyst comprises one or more active metals on an alkali metal silicate support or on an alkaline earth metal silicate support, wherein the support further comprises a silica enhancer and a support modifier.

### FIELD OF THE INVENTION

The present invention relates to catalysts, to processes for making catalysts, and to processes for manufacturing ethanol from a feedstock comprising acetic acid, ethyl acetate or a mixture thereof in the presence of the inventive catalysts. In particular, the present invention is related to catalysts comprising one or more active metals on an alkali metal silicate support or alkaline earth metal silicate support comprising a silica enhancer and a support modifier selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulosic materials, such as corn or sugar cane. Conventional methods for producing ethanol from petrochemical feed stocks, as well as from cellulosic materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol, which is suitable for fuels or human consumption. In addition, fermentation of starchy or cellulosic materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. The reduction of various carboxylic acids over metal oxides has been proposed by EP0175558 and U.S. Pat. No. 4,398,039. A summary of some of the developmental efforts for hydrogenation catalysts for conversion of various carboxylic acids is provided in Yokoyama, et al., “Carboxylic acids and derivatives” in: Fine Chemicals Through Heterogeneous Catalysis, 2001, 370-379.

U.S. Pat. No. 6,495,730 describes a process for hydrogenating carboxylic acid using a catalyst comprising activated carbon to support active metal species comprising ruthenium and tin. U.S. Pat. No. 6,204,417 describes another process for preparing aliphatic alcohols by hydrogenating aliphatic carboxylic acids or anhydrides or esters thereof or lactones in the presence of a catalyst comprising Pt and Re. U.S. Pat. No. 5,149,680 describes a process for the catalytic hydrogenation of carboxylic acids and their anhydrides to alcohols and/or esters in the presence of a catalyst containing a Group VIII metal, such as palladium, a metal capable of alloying with the Group VIII metal, and at least one of the metals rhenium, tungsten or molybdenum. U.S. Pat. No. 4,777,303 describes a process for the productions of alcohols by the hydrogenation of carboxylic acids in the presence of a catalyst that comprises a first component which is either molybdenum or tungsten and a second component which is a noble metal of Group VIII on a high surface area graphitized carbon. U.S. Pat. No. 4,804,791 describes another process for the production of alcohols by the hydrogenation of carboxylic acids in the presence of a catalyst comprising a noble metal of Group VIII and rhenium. U.S. Pat. No. 4,517,391 describes preparing ethanol by hydrogenating acetic acid under superatmospheric pressure and at elevated temperatures by a process using a predominantly cobalt-containing catalyst.

Existing processes suffer from a variety of issues impeding commercial viability including: (i) catalysts without requisite selectivity to ethanol; (ii) catalysts which are possibly prohibitively expensive and/or nonselective for the formation of ethanol and that produce undesirable by-products; (iii) required operating temperatures and pressures which are excessive; and/or (iv) insufficient catalyst life.

### SUMMARY OF THE INVENTION

In a first embodiment, the present invention is directed to a process for the formation of ethanol from acetic acid comprising contacting a feed stream containing acetic acid and hydrogen at an elevated temperature with a hydrogenating catalyst comprising one or more active metals on a support; wherein the support is an alkali metal silicate support or an alkaline earth metal silicate support; wherein the support further comprises a silica enhancer and a support modifier; and further wherein the silica enhancer is present from 1 to 10 wt. %, e.g., from 2 to 8 wt. %, and the support modifier is present from 10 to 30 wt. %, e.g., from 10 to 25 wt. % or from 15 to 25 wt. %, based on the total weight of the catalyst. The alkali metal silicate support may be selected from the group consisting of lithium silicate, sodium silicate and potassium silicate, lithium metasilicate, sodium metasilicate, potassium metasilicate, lithium orthosilicate, sodium orthosilicate, potassium orthosilicate. The alkaline earth metal silicate support may be selected from the group consisting of magnesium silicate, calcium silicate, strontium silicate, barium silicate, magnesium metasilicate, calcium metasilicate, strontium metasilicate, barium metasilicate, magnesium orthosilicate, calcium orthosilicate, strontium orthosilicate and barium orthosilicate. In some embodiments, the alkaline earth metal silicate support may be magnesium silicate calcium silicate, magnesium metasilicate or calcium metasilicate. The support may be present from 40 to 90 wt. %, e.g., from 50 to 80 wt. %, based on the total weight of the catalyst. The support modifier may be selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃. In some embodiments, the support modifier may be an oxide of tungsten. The silica enhancer may be selected from the group consisting of silica, pyrogenic silica, and high purity silica. The one or more active metals may be selected from the group consisting of cobalt, nickel, rhodium, palladium, osmium, iridium, platinum, titanium, zinc, chromium, rhenium, molybdenum, ruthenium, tin, vanadium, lanthanum, cerium, manganese, gold and combinations thereof. In preferred embodiments, the one or more active metals comprise platinum, tin, cobalt, or mixtures thereof. The one or more active metals may be present from 0.1 to 25 wt. %, based on the total weight of the catalyst. At least 45% of the acetic acid is consumed and selectivity to ethanol is at least 80%. Selectivity to ethyl acetate is less than 10%.

In a second embodiment, the present invention is directed to a process for the formation of ethanol from acetic acid comprising: contacting a feed stream containing acetic acid and hydrogen at an elevated temperature with a hydrogenating catalyst comprising one more active metals on an alkali metal silicate support or on an alkaline earth metal silicate support; wherein the support further comprises a silica enhancer and a support modifier; and further wherein the molar ratio of the support modifier to the silica enhancer is at least 8:1, based on the respective metals. The support modifier may be selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃. In some embodiments, the support modifier may be an oxide of tungsten.

In a third embodiment, the present invention is directed to a hydrogenation catalyst for converting acetic acid to ethanol comprising platinum, cobalt, and/or tin on an alkali metal silicate support or on an alkaline earth metal silicate support, wherein the support further comprises a support modifier and a silica enhancer in a molar ratio of at least 8:1, based on the respective metals. The support modifier may be selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃. In some embodiments, the support modifier may be an oxide of tungsten.

In a fourth embodiment, the present invention is directed to a hydrogenation catalyst for converting acetic acid to ethanol comprising one or more active metals on a calcium metasilicate support, wherein the support comprises from 1 to 10 wt. % of a silica enhancer and from 10 to 30 wt. % of a support modifier, based on the weight of the support. The support modifier may be selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃. In some embodiments, the support modifier may be an oxide of tungsten.

In a fifth embodiment, the present invention is directed to a process for producing a hydrogenation catalyst comprising providing an alkali metal silicate support or an alkaline earth metal silicate support; impregnating a support modifier support to form a modified support; adding a silica enhancer to the modified support to form a silica-containing modified support; impregnating one or more active metals on the silica-containing modified support to form an impregnated support; and heating and calcining the impregnated support under conditions effective to form and activate the hydrogenation catalyst.

In a sixth embodiment, the present invention is directed to a process for the formation of ethanol from acetic acid comprising contacting a feed stream containing acetic acid and hydrogen at an elevated temperature with a hydrogenating catalyst comprising one or more active metals on a calcium metasilicate support; wherein the support further comprises a silica enhancer and tungsten or an oxide thereof; and further wherein the silica enhancer is present from 1 to 10 wt. %, e.g., from 2 to 8 wt. %, and the tungsten or an oxide thereof is present from 10 to 30 wt. %, e.g., from 10 to 25 wt. % or from 15 to 25 wt. %, based on the total weight of the catalyst.

### DETAILED DESCRIPTION OF THE INVENTION

### Catalyst Composition

The present invention relates to a catalyst composition comprising one or more active metals on a support, wherein the support comprises an alkali metal silicate support or an alkaline earth metal silicate support. The support further comprises a silica enhancer and a support modifier. The support modifier may be an acidic or redox support modifier. In preferred embodiments, the support comprises calcium metasilicate, silica enhancer and tungsten or an oxide thereof. The present inventions also relates to processes for making the catalyst and to processes for hydrogenating acetic acid and/or ethyl acetate in the presence of the catalyst to form ethanol.

### Silica Enhancer

The alkali metal silicate support or alkaline earth metal silicate support comprises a silica enhancer. Without being bound by theory, the addition of a silica enhancer to the alkali metal or alkaline earth metal silicate support, in combination with a support modifier, unexpectedly increases in acetic acid conversion while also improving ethanol selectivity and decreasing selectivity to ethyl acetate. In one embodiment, the silica enhancer is selected from the group consisting of silica, pyrogenic silica, and high purity silica. The silica enhancer may be present from 1 to 10 wt. %, e.g., from 2 to 8 wt. %. In some embodiments, the silica enhancer is present at 7 wt. %.

The surface area of silica enhancer, e.g., silica, preferably is at least about 50 m²/g, e.g., at least about 100 m²/g, at least about 150 m²/g, at least about 200 m²/g or most preferably at least about 250 m²/g. In terms of ranges, the silica enhancer preferably has a surface area of from 50 to 600 m²/g, e.g., from 100 to 500 m²/g or from 100 to 300 m²/g. For purposes of the present specification, surface area refers to BET nitrogen surface area, meaning the surface area as determined by ASTM D6556-04, the entirety of which is incorporated herein by reference.

The silica enhancer also preferably has an average pore diameter of from 5 to 100 nm, e.g., from 5 to 30 nm, from 5 to 25 nm or from about 5 to 10 nm, as determined by mercury intrusion porosimetry, and an average pore volume of from 0.5 to 2.0 cm³/g, e.g., from 0.7 to 1.5 cm³/g or from about 0.8 to 1.3 cm³/g, as determined by mercury intrusion porosimetry.

The morphology of the silica enhancer, and hence of the resulting catalyst composition, may vary widely. In some exemplary embodiments, the morphology of silica enhancer and/or of the catalyst composition may be pellets, extrudates, spheres, spray dried microspheres, rings, pentarings, trilobes, quadrilobes, multi-lobal shapes, or flakes although cylindrical pellets are preferred. Preferably, the silica enhancer has a morphology that allows for a packing density of from 0.1 to 1.0 g/cm³, e.g., from 0.2 to 0.9 g/cm³ or from 0.3 to 0.8 g/cm³. In terms of size, the silica enhancer preferably has an average particle size, e.g., meaning the diameter for spherical particles or equivalent spherical diameter for non-spherical particles, of from 0.01 to 1.0 cm, e.g., from 0.1 to 0.7 cm or from 0.2 to 0.5 cm.

A preferred silica enhancer is SS61138 High Surface Area (HSA) Silica Catalyst Carrier from Saint-Gobain NorPro. The Saint-Gobain NorPro SS61138 silica contains approximately 95 wt. % high surface area silica; a surface area of about 250 m²/g; a median pore diameter of about 12 nm; an average pore volume of about 1.0 cm³/g as measured by mercury intrusion porosimetry and a packing density of about 0.352 g/cm³.

### Alkali Metal and Alkaline Earth Metal Silicate Support Materials

The catalysts of the present invention may be on a suitable silicate support material. The silicate support material may be obtained from natural occurring sources or may be synthetically made by combining an alkali metal or alkaline earth metal source with a silicon source. The silicate support material may be selected from the group consisting of alkali metal silicates, alkaline earth metal silicates, and mixtures thereof. In one embodiment, the silicate support has a molar ratio of the alkali metal or alkaline earth metal to silicon is from 0.5:1 to 2:1, e.g., from 0.6:1 to 1.5:1. For purposes of the present invention the term silicates also includes metasilicates and orthosilicates. In one embodiment the alkali metal silicate support is selected from the group consisting of lithium silicate, sodium silicate and potassium silicate, lithium metasilicate, sodium metasilicate, potassium metasilicate, lithium orthosilicate, sodium orthosilicate, potassium orthosilicate. In another embodiment the alkaline earth metal silicate support may be selected from the group consisting of magnesium silicate, calcium silicate, strontium silicate, barium silicate, magnesium metasilicate, calcium metasilicate, strontium metasilicate, barium metasilicate, magnesium orthosilicate, calcium orthosilicate, strontium orthosilicate and barium orthosilicate. Preferably, the silicate support material comprises magnesium silicate calcium silicate, magnesium metasilicate or calcium metasilicate, and more preferably calcium metasilicate.

In preferred embodiments, the alkali metal or alkaline earth metal silicate material is present in an amount from 40 to 90 wt. %, e.g., from 50 wt. % to 80 wt. %, based on the total weight of the catalyst.

The surface area of the silicate support material, preferably is at least about 50 m²/g, e.g., at least about 100 m²/g, at least about 150 m²/g, at least about 200 m²/g or most preferably at least about 250 m²/g. In terms of ranges, the support material preferably has a surface area of from 50 to 600 m²/g, e.g., from 100 to 500 m²/g or from 100 to 300 m²/g.

The silicate support material also preferably has an average pore diameter of from 5 to 100 nm, e.g., from 5 to 30 nm, from 5 to 25 nm or from about 5 to 10 nm, as determined by mercury intrusion porosimetry, and an average pore volume of from 0.5 to 2.0 cm³/g, e.g., from 0.7 to 1.5 cm³/g or from about 0.8 to 1.3 cm³/g, as determined by mercury intrusion porosimetry.

The morphology of the silicate support material, and hence of the resulting catalyst composition, may vary widely. In some exemplary embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the support material has a morphology that allows for a packing density of from 0.1 to 1.0 g/cm³, e.g., from 0.2 to 0.9 g/cm³ or from 0.3 to 0.8 g/cm³. In terms of size, the calcium metasilicate support material preferably has an average particle size, e.g., meaning the diameter for spherical particles or equivalent spherical diameter for non-spherical particles, of from 0.01 to 1.0 cm, e.g., from 0.1 to 0.7 cm or from 0.2 to 0.5 cm. Since the one or more active metal(s) that are disposed on or within the silicate support material are generally very small in size, those active metals should not substantially impact the size of the overall catalyst particles. Thus, the above particle sizes generally apply to both the size of the silicate support material as well as to the final catalyst particles. In terms of mesh size, the silicate support material may have a mesh size from 100 to 400, e.g., from 100 to 325, from 100 to 270 or from 100 to 200.

The silicate support material is preferably a high purity support material, meaning that it contains small amount of contaminants or additional components that may decrease the catalyst life, efficiency, conversion, selectivity to desired products, or productivity. The purity of the silicate support material may be from 90 to 99.9%, e.g., from 95 to 99.5%, or from 98 to 99%.

A preferred calcium metasilicate support material is Calcium silicate 372668 from Sigma-Aldrich®. The Sigma-Aldrich 372668 calcium silicate is 200 mesh and contains 99% calcium silicate and has a density of about 2.9 g/cm³.

The alkali metal or alkaline earth metal silicate support is generally basic and it may be necessary to introduce some acidity to the support material by adding the support modifier(s) discussed herein.

### Other Support Modifiers

The silicate support material comprises a support modifier. A support modifier may adjust the acidity of the silicate support material. In one embodiment, the support modifier may be present from 10 wt. % to 30 wt. %, e.g., from 10 wt. % to 25 wt. %, or from 15 wt. % to 25 wt. %, based on the total weight of the catalyst. In some embodiments, the support modifier may be present at 21 wt. %. In one embodiment, there is an excess molar amount of the support modifier to the silica enhancer. For example, the molar ratio of the support modifier to silica enhancer may at least 8:1, e.g., at least 10:1 or at least 15:1, based on the respective metals.

Support modifiers may adjust the acidity of the silicate support material. For example, the acid sites, e.g. Brønsted acid sites, on the silicate support material may be adjusted by the support modifier to favor selectivity to ethanol during the hydrogenation of acetic acid. The acidity of the silicate support material may adjust the number or the availability of Brønsted acid sites on the silicate support material. The silicate support material may also be adjusted by having the support modifier change the pKa of the silicate support material. Unless the context indicates otherwise, the acidity of a surface or the number of acid sites thereupon may be determined by the technique described in F. Delannay, Ed., “Characterization of Heterogeneous Catalysts”; Chapter III: Measurement of Acidity of Surfaces, p. 370-404; Marcel Dekker, Inc., N.Y. 1984, the entirety of which is incorporated herein by reference.

In some embodiments, the support modifier may be an acidic modifier or redox modifier that increases the acidity of the catalyst. Suitable support modifiers may be selected from the group consisting of: oxides of Group IVB metals, oxides of Group VB metals, oxides of Group VIB metals, oxides of Group VIIB metals, oxides of Group VIII metals, aluminum oxides, and mixtures thereof. Support modifiers include those selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃. In one embodiment, the support modifier is selected from the group consisting of WO₃, MoO₃, V₂O₅, Nb₂O₅, and Ta₂O₅. Preferably the modifier comprises tungsten or an oxide thereof.

### Active Metals

The catalyst compositions of the present invention may comprise one or more active metals. The one or more active metals may comprise a first metal and optionally one or more of a second metal, a third metal or any number of additional metals. Preferred metal combinations for some exemplary catalyst compositions include platinum/tin, platinum/tin/cobalt, platinum/ruthenium, platinum/rhenium, palladium/ruthenium, palladium/rhenium, cobalt/palladium, cobalt/palladium/tin, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron. Exemplary catalysts are further described in U.S. Pat. No. 7,608,744 and U.S. Pub. No. 2010/0029995, the entireties of which are incorporated herein by reference. In another embodiment, the catalyst comprises a Co/Mo/S catalyst of the type described in U.S. Pub. No. 2009/0069609, the entirety of which is incorporated herein by reference.

In one embodiment, the catalyst comprises a first metal selected from the group consisting of cobalt, nickel, rhodium, palladium, osmium, iridium, platinum, titanium, zinc, chromium, rhenium, ruthenium, molybdenum, tin, vanadium, lanthanum, cerium, manganese, gold and combinations thereof.

Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

The catalyst may also further comprise a second metal, which typically would function as a promoter. The second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is tin.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 7.5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.1 to 20 wt. %, e.g., from 0.5 to 15 wt. %, or from 1 to 10 wt. %.

The one or more active metals may be present from 0.1 to 25 wt. %, e.g., 0.5 to 15 wt. %, or from 0.5 wt. % to 10 wt. %. For purposes of the present specification, unless otherwise indicated, weight percent of the one or more active metals is based on the total weight the catalyst including metal and support.

In some embodiments, when the one or more active metals comprise platinum and tin, the catalyst may comprise from 0.1 wt. % to 20 wt. %. platinum, e.g., from 0.2 wt. % to 5 wt. % platinum or from 0.5 wt. % to 2 wt. % platinum. The catalyst composition may comprise from 0.1 wt. % to 20 wt. % tin, e.g., from 0.2 wt. % to 10 wt. % tin or from 0.5 wt. % to 7.5 wt. % tin. The catalyst composition may further comprise from 0.1 to 20 wt. % cobalt, e.g., from 0.5 to 15 wt. %, or from 1 to 10 wt. %.

### Process for Preparing the Catalyst Compositions

The present invention also relates to processes for making the catalyst. Generally, the hydrogenation catalyst is prepared by impregnating a support modifier onto the silicate support material to form a modified support. A silica enhancer, e.g., silica, is then added to form a silica-containing modified support. The silica enhancer may be added as a powder and mixed with the silicate support material. The silica enhancer may be added prior to adding the support modifier. The silica-containing modified support is then impregnated with one or more active metals to form the hydrogenation catalyst. The hydrogenation catalyst is then heated and calcined, under conditions effective to activate the catalyst.

One or more support modifiers are added to the silicate support material by mixing or through impregnation. Powdered materials of the modified supports or a precursor thereto may pelletized, crushed and sieved and added to the silicate support material. The use of a solvent, such as water, glacial acetic acid, a strong acid such as hydrochloric acid, nitric acid, or sulfuric acid, or an organic solvent, may be preferred. The resulting mixture may be stirred and added to additional support material using, for example, incipient wetness techniques. Capillary action then draws the additional support modifiers into the pores in the silicate support material. The silicate support material containing a support modifier and silica enhancer can then be formed by drying to drive off water and any volatile components within the support solution and depositing the support modifier on the support material. Drying may occur, for example, at a temperature of from 50° C. to 300° C., e.g., from 100° C. to 200° C. or about 120° C., optionally for a period of from 1 to 24 hours, e.g., from 3 to 15 hours or from 6 to 12 hours.

Once formed, the modified supports may be shaped into particles having the desired size distribution, e.g., to form particles having an average particle size in the range of from 0.2 to 0.4 cm. The supports may be extruded, pelletized, tabletized, pressed, crushed or sieved to the desired size distribution. Any of the known methods to shape the support materials into desired size distribution can be employed.

In a preferred method of preparing the catalyst, the one or more active metals are impregnated onto the support. In some embodiments, when the one or more active metals are platinum and tin, a precursor of platinum preferably is used in the metal impregnation step, such as a water soluble compound or water dispersible compound/complex that includes platinum. Depending on the metal precursor employed, the use of a solvent, such as water, glacial acetic acid or an organic solvent, may be preferred. The tin precursor also preferably is impregnated on the support. The order of the platinum, tin, and promoter metal precursor may vary and preferably the metals may be co-impregnated. Preferably, the tin is impregnated first and the support is calcined. The platinum is then added and the support is again calcined.

Impregnation occurs by adding, optionally drop wise, the platinum precursor, the tin precursor and/or the promoter metal precursors, preferably in suspension or solution, to the dry support. The resulting mixture may then be heated, e.g., optionally under vacuum, in order to remove the solvent. Additional drying and calcining may then be performed, optionally with ramped heating to form the final catalyst composition. Upon heating and/or the application of vacuum, the metal(s) of the metal precursor(s) preferably decompose into their elemental (or oxide) form. In some cases, the completion of removal of the liquid carrier, e.g., water, may not take place until the catalyst is placed into use and calcined, e.g., subjected to the high temperatures encountered during operation. During the calcination step, or at least during the initial phase of use of the catalyst, such compounds are converted into a catalytically active form of the metal or a catalytically active oxide thereof.

Impregnation of the one or more active metals on the support may occur simultaneously (co-impregnation) or sequentially. In simultaneous impregnation, the precursors are mixed together and added to the support together, followed by drying and calcination to form the final catalyst composition. With simultaneous impregnation, it may be desired to employ a dispersion agent, surfactant, or solubilizing agent, e.g., ammonium oxalate, to facilitate the dispersing or solubilizing of the precursors in the event the precursors are incompatible with the desired solvent, e.g., water.

In sequential impregnation, when the one or more active metals are platinum and tin, the tin precursor is first added to the support followed by drying and calcining, and the resulting material is then impregnated with the platinum precursor followed by impregnated with the promoter metal precursor, and additional drying and calcining step to form the final catalyst composition. Of course, combinations of sequential and simultaneous impregnation may be employed if desired.

Suitable metal precursors include, for example, metal halides, amine solubilized metal hydroxides, metal nitrates or metal oxalates. For example, suitable compounds for platinum precursors include chloroplatinic acid, ammonium chloroplatinate, amine solubilized platinum hydroxide, platinum nitrate, platinum tetra ammonium nitrate, platinum chloride, platinum oxalate, and sodium platinum chloride. Generally, both from the point of view of economics and environmental aspects, aqueous solutions of soluble compounds of platinum are preferred. A particularly preferred precursor to platinum is platinum ammonium nitrate, Pt(NH₃)₄(NO₄)₂. Calcining of the solution with the support and active metal may occur, for example, at a temperature of from 250° C. to 800° C., e.g., from 300 to 700° C. or about 500° C., optionally for a period of from 1 to 12 hours, e.g., from 2 to 10 hours, from 4 to 8 hours or about 6 hours.

As an example, PtSn/CaSiO₃—WO₃—SiO₂ may be prepared by a mixing CaSiO₃ and WO₃ to form a modified support. A silica enhancer is then mixed with the modified support. Next there is a sequential or co-impregnation with Pt(NH₃)₄(NO₄)₂ and Sn(AcO)₂, or other suitable precursor. Again, each impregnation step of Pt and Sn may be followed by drying and calcination steps as necessary. In most cases, the impregnation may be carried out using metal nitrate solutions. However, various other soluble salts, which upon calcination release metal ions, can also be used. Examples of other suitable metal salts for impregnation include, metal acids, such as perrhenic acid solution, metal oxalates, and the like.

### Use of Catalyst to Hydrogenate Acetic Acid

One advantage of catalysts of the present invention is the stability or activity of the catalyst for producing ethanol. Accordingly, it can be appreciated that the catalysts of the present invention are fully capable of being used in commercial scale industrial applications for hydrogenation of acetic acid, particularly in the production of ethanol. In particular, it is possible to achieve such a degree of stability such that catalyst activity will have a rate of productivity decline that is less than 6% per 100 hours of catalyst usage, e.g., less than 3% per 100 hours or less than 1.5% per 100 hours. Preferably, the rate of productivity decline is determined once the catalyst has achieved steady-state conditions.

In one embodiment there is a process for producing ethanol by hydrogenating feedstock comprising compounds selected from the group consisting of acetic acid, ethyl acetate and mixtures thereof in the presence of the catalyst. One particular preferred reaction is to make ethanol from acetic acid. The hydrogenation reaction may be represented as follows:

HOAc+2H₂→EtOH+H₂O

The raw materials, acetic acid and hydrogen, fed to the primary reactor used in connection with the process of this invention may be derived from any suitable source including natural gas, petroleum, coal, biomass, and so forth. As examples, acetic acid may be produced via methanol carbonylation, acetaldehyde oxidation, ethane oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The acetic acid fed to the hydrogenation reactor may also comprise other carboxylic acids and anhydrides, as well as aldehyde and/or ketones, such as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the hydrogenation reactor without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

The reactor, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation in the reactor may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, from 100 kPa to 2100 kPa or from 200 kPa to 2100 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹. The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 18:1 to 8:1. Most preferably, the molar ratio of hydrogen to acetic acid is greater than 2:1, e.g., greater than 4:1 or greater than 8:1. Generally, the reactor may use an excess of hydrogen, while the secondary hydrogenation reactor may use a sufficient amount of hydrogen as necessary to hydrogenate the impurities. In one aspect, a portion of the excess hydrogen from the reactor is directed to the secondary reactor for hydrogenation. In some optional embodiments, the secondary reactor could be operated at a higher pressure than the hydrogenation reactor and a high pressure gas stream comprising hydrogen may be separated from the secondary reactor liquid product in an adiabatic pressure reduction vessel, and the gas stream could be directed to the hydrogenation reactor system.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

In particular, the hydrogenation of acetic acid may achieve favorable conversion of acetic acid and favorable selectivity and productivity to ethanol in the primary reactor. For purposes of the present invention, the term “conversion” refers to the amount of acetic acid in the feed that is converted to a compound other than acetic acid. Conversion is expressed as a percentage based on acetic acid in the feed. The conversion may be at least 45%, e.g., at least 50%, at least 55% or at least 60%. Although catalysts that have high conversions are desirable, such as at least 60%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. In one embodiment, catalyst selectivity to ethanol is at least 80%, e.g., at least 83%, or at least 85%. Preferably, the selectivity to ethanol is at least 85%, e.g., at least 86% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

An additional undesirable product that may be formed during the hydrogenation process is ethyl acetate, which over time, may build up in the reactor and may decrease process efficiency and ethanol yield. In some embodiments, the selectivity to ethyl acetate is less than 10%.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

In various embodiments of the present invention, the crude ethanol product produced by the reactor, before any subsequent processing, such as purification and separation, will typically comprise unreacted acetic acid, ethanol and water. Exemplary compositional ranges for the crude ethanol product are provided in Table 1. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 1 to 25 | 3 to 20 | 5 to 18 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product may comprise acetic acid in an amount less than 20 wt. %, e.g., of less than 15 wt. %, less than 10 wt. % or less than 5 wt. %. In terms of ranges, the acetic acid concentration of Table 1 may range from 0.1 wt. % to 20 wt. %, e.g., 0.2 wt. % to 15 wt. %, from 0.5 wt. % to 10 wt. % or from 1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

An ethanol product may be recovered from the crude ethanol product produced by the reactor using the catalyst of the present invention may be recovered using several different techniques.

The ethanol product may be an industrial grade ethanol comprising from 75 to 96 wt. % ethanol, e.g., from 80 to 96 wt. % or from 85 to 96 wt. % ethanol, based on the total weight of the ethanol product. In some embodiments, when further water separation is used, the ethanol product preferably contains ethanol in an amount that is greater than 97 wt. %, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, ethyl benzene, aldehydes, butadiene, and higher alcohols, especially butanol. In the production of ethyl acetate, the finished ethanol composition may be esterified with acetic acid. In another application, the finished ethanol composition may be dehydrated to produce ethylene. Any known dehydration catalyst, such as zeolite catalysts or phosphotungstic acid calaysts, can be employed to dehydrate ethanol, as described in U.S. Pub. Nos. 2010/0030002 and 2010/0030001 and WO2010146332, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Y in U.S. Pat. No. 3,130,007, the entireties of which are hereby incorporated herein by reference.

The following examples describe the catalyst and process of this invention.

### EXAMPLES

### Catalyst Examples A to D

To prepare Example A, a catalyst support containing CaSiO₃—WO₃(14 wt. %) was prepared as follows. CaSiO₃ support are placed in a 250 mL round-bottomed flask. A solution of ammonium metatungstate dissolved in H₂O was used to impregnate the support using an incipient wetness technique. The impregnated support was then dried on a rotor-evaporator and dried at 120° C. for six hours in an oven.

The modified support was then calcined under flowing air at 550° C./6 hours.

Sn(C₂O₄) was added, followed sequentially by adding Pt(C₂O₄) to form the Pt/Sn-containing Catalysts A-D. Sn was first added by wet impregnation using 375.1 g/mL of the tin precursor solution. 8M HNO₃ was used as the solvent. The impregnation volume was 1750 μL for adding the tin precursor solution. The same process was followed for Pt, using 102.3 g/mL of the platinum precursor solution. 8M HNO₃ was used as the solvent. The impregnation volume was 1750 μL for adding the platinum precursor solution.

The same process was followed for Example B, except that the support contains CaSiO₃—WO₃(21 wt. %) A solution ammonium metatungstate dissolved in H₂O was used to impregnate the support using an incipient wetness technique.

To prepare Example C, the process of Example A was used, except that prior to modifying the support with support modifier, powdered silica was added to the powdered CaSiO₃ support and mixed.

To prepare Example D, the process of Example B was used, except that prior to modifying the support with support modifier, powdered silica was added to the powdered CaSiO₃ support and mixed.

Table 2 summarizes the catalysts prepared.

TABLE 2
IMPREGNATED CATALYSTS A to D

| | | First | Second |
|---|---|---|---|
| Catalyst | Support | Metal | Metal |
| A | CaSiO₃—WO₃ (14 wt. %) | Pt | Sn |
| B | CaSiO₃—WO₃ (21 wt. %) | Pt | Sn |
| C | CaSiO₃—WO₃ (14 wt. %) SiO₂(7 wt. %) | Pt | Sn |
| D | CaSiO₃—WO₃ (21 wt. %) SiO₂(7 wt. %) | Pt | Sn |

Catalysts A through D are placed in separate reactor vessels and dried by heating at 120° C. Feedstock acetic acid vapor was then charged to the reactor vessels along with hydrogen and helium as a carrier gas with an average combined gas hourly space velocity (GHSV) of 2430 hr⁻¹, temperature of 250° C., pressure of 2500 kPa, and mole ratio of hydrogen to acetic acid of 8:1. Product samples are taken and analyzed to determine conversion and selectivity. Analysis of the products was carried out by online GC. A three channel compact GC equipped with one flame ionization detector (FID) and 2 thermal conducting detectors (TCD) was used to analyze the feedstock reactant and reaction products. The front channel was equipped with an FID and a CP-Sil 5 (20 m)+WaxFFap (5 m) column and was used to quantify: acetaldehyde; ethanol; acetone; methyl acetate; vinyl acetate; ethyl acetate; acetic acid; ethylene glycol diacetate; ethylene glycol; ethylidene diacetate; and paraldehyde. The middle channel was equipped with a TCD and Porabond Q column and was used to quantify: CO₂; ethylene; and ethane. The back channel was equipped with a TCD and molecular sieve 5A column and was used to quantify: helium; hydrogen; nitrogen; methane; and carbon monoxide.

Table 3 summarizes the conversion of acetic acid and selectivity to ethanol and ethyl acetate for each of Catalysts A to D.

TABLE 3
ACETIC ACID CONVERSION AND ETHANOL
SELECTIVITY FOR CATALYSTS A TO D

| | Acetic Acid | Ethanol | Ethyl Acetate |
|---|---|---|---|
| | Conversion | Selectivity | Selectivity |
| Catalyst | (%) | (%) | (%) |
| A | 39 | 85 | 12 |
| B | 37 | 86 | 11 |
| C | 43 | 85 | 12 |
| D | 64 | 89 | 9 |

As shown in Table 3, unexpectedly the silica enhanced Catalysts C and D demonstrate an improvement in acetic acid conversion over Catalysts A and B. Ethyl acetate selectivity also decreased with Catalyst D.

While the invention has been described in detail, modifications within the spirit and scope of the invention will be readily apparent to those skilled in the art. All publications and references discussed above are incorporated herein by reference. In addition, it should be understood that aspects of the invention and portions of various embodiments and various features recited may be combined or interchanged either in whole or in part. In the foregoing descriptions of the various embodiments, those embodiments which refer to another embodiment may be appropriately combined with other embodiments as will be appreciated by one skilled in the art. Furthermore, those skilled in the art will appreciate that the foregoing description is by way of example only, and is not intended to limit the invention.

We claim:

1. A process for the formation of ethanol from acetic acid comprising:
contacting a feed stream containing acetic acid and hydrogen at an elevated temperature with a hydrogenating catalyst comprising one or more active metals on a support;
wherein the support is an alkali metal silicate support or an alkaline earth metal silicate support;
wherein the support further comprises a silica enhancer and a support modifier; and
further wherein the silica enhancer is present from 1 to 10 wt. % and the support modifier is present from 10 to 30 wt. %, based on the total weight of the catalyst.

2. The process of claim 1, wherein the alkali metal silicate support is selected from the group consisting of lithium silicate, sodium silicate and potassium silicate, lithium metasilicate, sodium metasilicate, potassium metasilicate, lithium orthosilicate, sodium orthosilicate, potassium orthosilicate.

3. The process of claim 1, wherein the alkaline earth metal silicate support is selected from the group consisting of magnesium silicate, calcium silicate, strontium silicate, barium silicate, magnesium metasilicate, calcium metasilicate, strontium metasilicate, barium metasilicate, magnesium orthosilicate, calcium orthosilicate, strontium orthosilicate and barium orthosilicate.

4. The process of claim 3, wherein the alkaline earth metal silicate support is calcium metasilicate.

5. The process of claim 1, wherein the support modifier is selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃.

6. The process of claim 1, wherein the support modifier is an oxide of tungsten.

7. The process of claim 1, wherein the alkali metal silicate support or alkaline earth metal silicate support is present from 40 to 90 wt. %, based on the weight of the support.

8. The process of claim 1, wherein the support modifier is present from 15 to 25 wt. %, based on the weight of the support.

9. The process of claim 1, wherein the silica enhancer is selected from the group consisting of silica, pyrogenic silica, and high purity silica.

10. The process of claim 1, wherein the one or more active metals are selected from the group consisting of cobalt, nickel, rhodium, palladium, osmium, iridium, platinum, titanium, zinc, chromium, rhenium, molybdenum, ruthenium, tin, vanadium, lanthanum, cerium, manganese, gold and combinations thereof.

11. The process of claim 1, wherein the one or more active metals comprise platinum, tin, cobalt, or mixtures thereof.

12. The process of claim 1, wherein the one or more active metals are present in an amount from 0.1 to 25 wt. %, based on the total weight of the catalyst.

13. The process of claim 1, wherein at least 45% of the acetic acid is consumed.

14. The process of claim 1, wherein selectivity of acetic acid to ethanol is at least 80%.

15. The process of claim 1, wherein selectivity of acetic acid to ethyl acetate is less than 10%.

16. A process for the formation of ethanol from acetic acid comprising:
contacting a feed stream containing acetic acid and hydrogen at an elevated temperature with a hydrogenating catalyst comprising one more active metals on an alkali metal silicate support or on an alkaline earth metal silicate support;
wherein the support further comprises a silica enhancer and a support modifier; and
further wherein the molar ratio of the support modifier to the silica enhancer is at least 8:1.

17. The process of claim 16, wherein the support modifier is selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃.

18. The process of claim 16, wherein the support modifier is an oxide of tungsten.

19. A hydrogenation catalyst for converting acetic acid to ethanol comprising platinum, cobalt, and/or tin on an alkali metal silicate support or on an alkaline earth metal silicate support, wherein the support further comprises a support modifier and a silica enhancer in a molar ratio of at least 8:1.

20. A hydrogenation catalyst for converting acetic acid to ethanol comprising one or more active metals on a calcium metasilicate support,
further wherein the support comprises from 1 to 10 wt. % of a silica enhancer and from 10 to 30 wt. % of a support modifier, based on the weight of the support.

21. A process for producing a hydrogenation catalyst comprising:
providing an alkali metal silicate support or an alkaline earth metal silicate support;
impregnating a support modifier support to form a modified support;
adding a silica enhancer to the modified support to form a silica-containing modified support;
impregnating one or more active metals on the silica-containing modified support to form an impregnated support; and
heating and calcining the impregnated support under conditions effective to form and activate the hydrogenation catalyst.
